(19) United States
(12) Patent Application Publication
(10) Pub. No.: US 2005/0273010 A1
(43) Pub. Date: Dec. 8, 2005

(54) Method and system for ultrasound contrast-imaging

(76) Inventors: William Tao Shi, Elm Grove, WI (US); Richard Yung Chiao, Menomonee Falls, WI (US); Anne Lindsay Hall, New Berlin, WI (US)

Correspondence Address: Dean D. Small;Armstrong Teasdale LLP, Suite 2600, One Metropolitan Square, St. Louis, MO, 63102, US

(21) Appl. No.: 10/860,187

(22) Filed: Jun. 2, 2004

(51) Int. Cl. A61B 8/14; A61B 8/02

(52) U.S. Cl. 600/458; 600/447

(57) ABSTRACT

Methods and systems for controlling an ultrasound system are provided. The method includes transmitting an imaging pulse sequence and transmitting at least one modification pulse between successive pulses in the imaging pulse sequence. The modification pulse is configured to change a contrast agent within an object being imaged by an ultrasound system.

### BACKGROUND OF THE INVENTION

The present invention relates generally to ultrasound imaging systems. More particularly, the present invention relates to methods and systems for contrast-imaging in ultrasound imaging systems.

Medical ultrasound systems may be used to study, for example, anatomical structures, detect anomalies in tissues and measure blood flow within the body. Ultrasound systems typically comprise a transducer, which is used for transmitting pulses of ultrasound waves, known as imaging pulses, into the body. Acoustic echo signals are generated at interfaces in the body in response to these waves. These echo signals are received by the transducer and transformed into an electrical signal that can be used to produce an image of the body part under examination. This image may be displayed on a display device.

In typical ultrasound systems, the amplitude of reflected ultrasound waves is detected and measured as a function of time to construct dynamic or static images of structures within a body. These ultrasound systems, however, have a limited ability to image blood flow within small and deep lying blood vessels in the body.

The use of administrable ultrasound contrast agents has improved the imaging of blood flow, particularly within small and deep lying blood vessels and capillaries. Thus, improved ultrasound images of organs and surrounding tissue are obtained by introducing contrast agents into the bloodstream and organs to be investigated. The contrast agents typically contain microbubbles stabilized with additional material (e.g., as Albumin, Polymer, Phospholipid, Liposomes, Galactose, etc.) on their surface. Such microbubbles show nonlinear behavior upon interaction with ultrasound waves.

When contrast agent microbubbles are irradiated with ultrasound waves, the microbubbles oscillate or resonate nonlinearly, returning a spectrum of echo signals including those at the second harmonic of the transmitted frequency. The strong harmonic echo components uniquely distinguish echoes returning from the microbubbles. Therefore, an improved contrast in the image may be achieved. The amount of ultrasound energy that is scattered and returned by contrast agents is mainly a function of microbubble size and surface properties (e.g., surface elasticity and viscosity) and the frequency and pressure of the ultrasound wave. For a given ultrasound frequency, there is a corresponding microbubble resonance size that is unique for each contrast agent. The effective scattering strength of microbubbles reaches a peak when microbubbles resonant at the incident ultrasound frequency. A significant difference in the harmonic component strength of the reflected signals due to the microbubbles and tissue makes it possible to obtain an image with higher contrast.

Although administration of contrast agents generally improves imaging, it may reduce contrast between the region of interest and its surrounding anatomical structures. Microbubbles that scatter ultrasound can greatly dampen the ultrasound waves, thereby affecting echo intensity and the image produced by targeted anatomical structures. High intensity ultrasound pulses may be transmitted to ensure that microbubble size, after modification, has reached a state where the microbubble can resonate, thereby improving contrast. However, the microbubbles may get destroyed and, thus, real-time imaging may not be possible.

Thus, known methods for providing ultrasound imaging employing contrast agents have several limitations that may affect the acquired and displayed image.

### BRIEF DESCRIPTION OF THE INVENTION

In one exemplary embodiment, a method for controlling an ultrasound system is provided. The method includes transmitting a nonlinear contrast imaging pulse sequence and transmitting at least one modification pulse between successive pulses in the nonlinear contrast imaging pulse sequence. The modification pulse is configured to change a contrast agent within an object being imaged by an ultrasound system.

In another exemplary embodiment, an ultrasound system is provided. The ultrasound system includes an ultrasound probe having at least one transducer for transmitting pulses to an object and a controller configured to transmit at least one modification pulse between successive pulses in a nonlinear contrast imaging pulse sequence. The modification pulse changes a contrast agent within an object being imaged by the ultrasound probe.

### BRIEF DESCRIPTION OF THE DRAWINGS

FIG. 1 is a flowchart illustrating a process for transmitting imaging and modification pulses in accordance with an exemplary embodiment of the present invention.

FIG. 2 is a flowchart illustrating a process performed after transmission of imaging and modification pulses and for generating an image in accordance with an exemplary embodiment of the present invention.

FIG. 3 is a signal pulse diagram showing the pulse sequencing employed in a typical pulse inversion process for contrast-imaging using an ultrasound system.

FIG. 4 is a signal pulse diagram showing the pulse sequencing for modification pulse enhanced contrast-imaging using an ultrasound system in accordance with an exemplary embodiment of the present invention.

FIG. 5 is a block diagram of an ultrasound system in accordance with an exemplary embodiment of the present invention for transmitting modification pulses.

### DETAILED DESCRIPTION OF THE INVENTION

Various embodiments of the present invention provide methods and systems for improving the contrast harmonic signals generated in response to nonlinear imaging pulses in an ultrasound imaging system. In one embodiment, a modification pulse is provided with the nonlinear imaging pulses. Various embodiments of the present invention generate enhanced images of anatomical structures and blood flow in a body when using ultrasound contrast agents including microbubbles by modifying, for example, the physical and acoustic properties of microbubbles using one or more modification pulses.

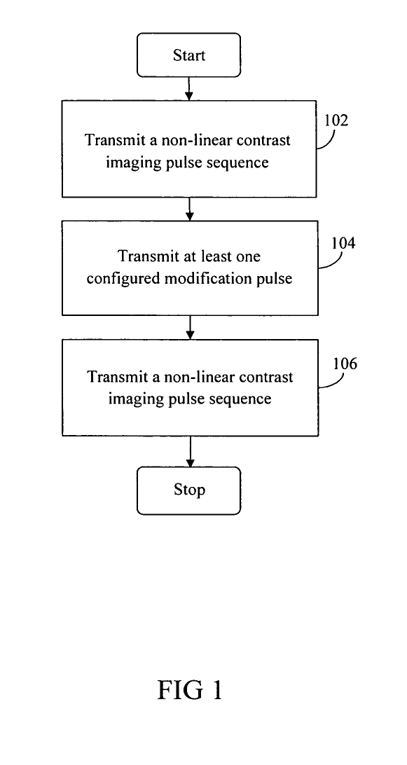

FIG. 1 is a flowchart illustrating a process for transmitting imaging and modification pulses in accordance with an exemplary embodiment of the present invention. Upon initiation of an ultrasound imaging process, an imaging pulse sequence is transmitted at 102. After transmitting the imaging pulse at 102, at least one modification pulse is transmitted at 104. The modification pulse initiates a change (e.g., an instantaneous or continuing change) in the properties of the microbubbles, such as, for example, a change in the acoustic or physical properties of the microbubbles (e.g., microbubble size or surface property). After transmitting the modification pulse at 104, a second imaging pulse sequence is transmitted at 106. The imaging pulse sequence at 106 is generally different from that at 102. The imaging pulse sequences at 102 and at 106 can be combined to extract nonlinear contrast signals at both the fundamental and the second harmonic frequency bands, (e.g., a short pulse A at 102 and an inverted copy of the pulse A at 106) using known pulse-inversion harmonic imaging.

In various embodiments of the present invention, the modification pulse is configured to change the acoustic and/or physical properties of the contrast agent microbubbles without affecting the surrounding environment (e.g., tissue in a body). In an embodiment, the modification pulse is used to make the contrast agent microbubbles more flexible. In another embodiment, the encapsulation of the contrast agent microbubbles is removed by utilizing the modification pulse, thereby providing free microbubbles. The increase in flexibility and providing of free microbubbles increases scattering capabilities of the contrast agent microbubbles. The size of the contrast agent microbubbles, before modification, ranges generally from sub-micron size to about ten microns. In various embodiments, the size of the contrast agent microbubbles is increased to a resonant size that is in the range of one to ten microns, which is similar to or smaller than the size of blood cells. The modification pulse is also configured in various embodiments to increase the intensity of the second harmonic signal from the contrast agent microbubbles. It should be noted that the modification pulse does not change the acoustic properties of, for example, tissues in a human body being imaged.

In another embodiment, the modification pulse is configured to change the resonant frequency of the contrast agent microbubbles rather than changing the size of the microbubbles. The change in resonant frequency of the contrast agent microbubbles is performed such that the resonant frequency matches the imaging pulse sequence frequency. Essentially, the modification pulse changes (e.g., shifts) the resonant frequency of the microbubbles to an imaging frequency. This increases the contrast achieved while using the echo response of the imaging pulse sequences.

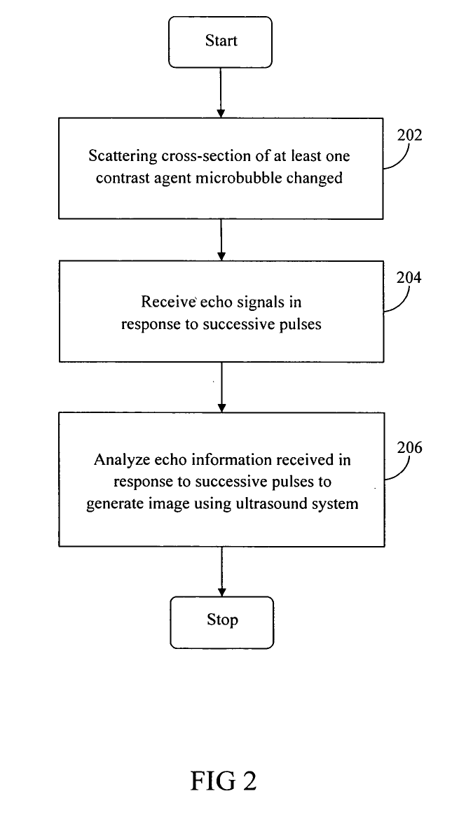

It should be noted that the modification pulse also may be configured to be transmitted, for example, between successive pulses of an imaging pulse sequence. In one embodiment, transmission of the imaging pulses increases the size of the microbubbles. This is a direct result of the compression and rarefaction occurring because of the nonlinear imaging pulses. This decrease and increase to a resonant size of the microbubbles is not symmetric (e.g., microbubbles once expanded do not return to their original size after compression). A harmonic signal generation in response to the incident nonlinear imaging pulses results. Transmission of the modification pulse at 104 increases the size of the microbubbles to a resonance size, thereby improving the imaging performance of the ultrasound system. In other embodiments, transmission of the modification pulse at 104 may change other properties of the microbubbles including, for example, the surface properties of the microbubbles (e.g., elasticity). Based on the transmitted pulses, an image of a body part under examination is generated. The process for generation of images is further described in detail in connection with FIG. 2.

FIG. 2 is a flowchart illustrating a process performed after transmission of imaging and modification pulses and for generating an image in accordance with an exemplary embodiment of the present invention. At 202, the scatterability or scattering cross-section of at least one contrast agent microbubble is changed in response to the incident nonlinear imaging pulse sequences and modification pulses as described herein. The modified microbubbles generate echoes corresponding to successive incident pulses. These echoes are then received by the ultrasound system at 204 and separation of harmonics from the fundamental frequency is performed. In another embodiment, the nonlinear portion of the amplitude of these echoes in the transmit frequency band is obtained when using imaging pulses with different amplitudes within imaging pulse sequences.

An analysis of echo signals is performed at 206. In various embodiments, the analysis includes separating harmonic and linear components, enhancing the harmonic component and generating an ultrasound image by utilizing the enhanced harmonic component. Separation of harmonic components from the received signals is performed using suitable filters.

In various other embodiments of the present invention, the separation of harmonic components may be performed by utilizing a processor. In these embodiments, a transducer within the ultrasound system converts the echo signals to electrical signals. These electrical signals are then converted to a digital form by employing an analog-to-digital converter. The processor processes the digital data for separating harmonic components using a suitable algorithm as is known. The separated harmonic signals, received in response to the imaging and modification pulse, are then added for enhancing the harmonic component of the signal for generating enhanced contrast images using the ultrasound imaging system.

In an exemplary embodiment of the present invention, echoes received in response to the imaging pulses are used for generating images using an ultrasound imaging system. The modification pulse is used for generating a weighted image (e.g., for enhancing the intensity of reflected echo signals by changing the acoustic and physical properties of the contrast agent microbubbles) and the response generated as a result of this pulse is thus not used in generating the images. In another exemplary embodiment of the present invention, echoes received in response to the imaging pulses and the modification pulse are used for generating images using the ultrasound imaging system.

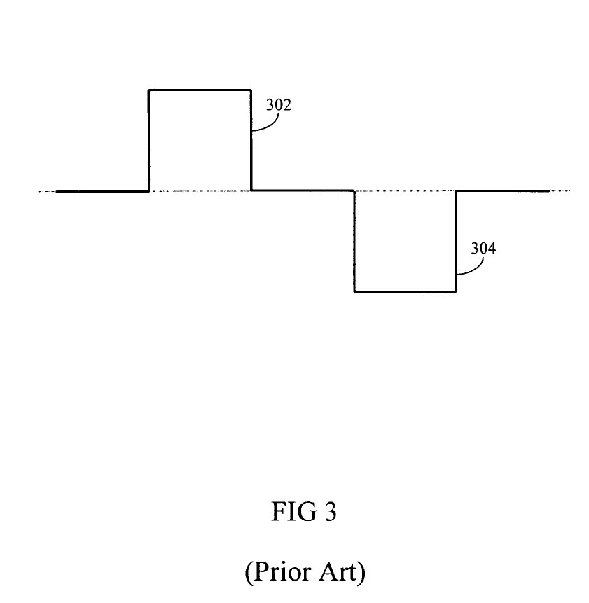

FIG. 3 is a signal pulse diagram showing the pulse sequencing employed in a typical pulse inversion technique for contrast-imaging using an ultrasound system. A first imaging pulse 302 is transmitted followed by a second imaging pulse 304. The second imaging pulse 304 is similar to first imaging pulse 302 (e.g., same amplitude and duration) except that it is out of phase with the first imaging pulse 302 (e.g., 180 degrees out of phase). This technique of transmitting two similar pulses 302 and 304 that are out of phase ensures that the linear component of the echo signals in response to these two pulses 302 and 304 also are out of phase. When adding these echoes, the two linear components of the pulses 302 and 304 cancel out and no linear component of the echo signal remains.

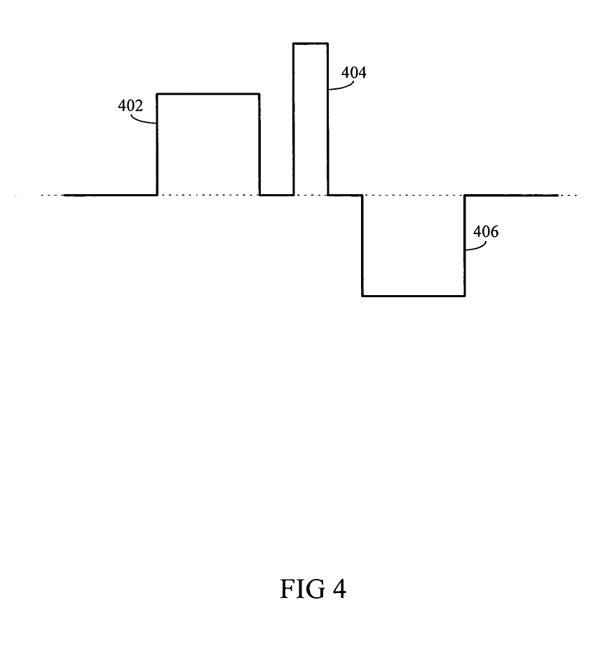

FIG. 4 is a signal pulse diagram showing the pulse sequencing for modification pulse enhanced contrast-imaging using an ultrasound system in accordance with an exemplary embodiment of the present invention. A first imaging pulse 402 and a second imaging pulse 406 are transmitted in a manner similar to that described in connection with FIG. 3. The two pulses 402 and 406 are similar (e.g., same amplitude and duration) except that the pulses 402 and 406 are out of phase with each other. Modification pulse 404 is introduced between the first imaging pulse 402 and second imaging pulse 406. In various embodiments, the amplitude and frequency of the modification pulse vary depending on the physical and acoustic properties of the contrast agent microbubbles being used. In various embodiments, the modification pulse has a frequency of 2 MHz with a tone burst of two to sixteen cycles and a pressure ranging from 0.4 to 1.6 Mega Pascal. In the various embodiments, the range of separation in time of the modification pulse from the two successive imaging pulses varies from a few microseconds to a few hundred microseconds. The modification pulse may be transmitted from the same transducer transmitting the imaging pulses or may be transmitted from a separate unit different from the transducer transmitting the imaging pulses.

Modification pulse 404 changes contrast agent microbubbles, for example, changes the physical and/or acoustic property of the microbubble. For example, modification pulse 404 may increase the contrast agent microbubble size to about equal to and or above a resonance size of the microbubble. The introduction of modification pulse 404 causes an expansion of the contrast agent microbubbles and thereby increases the effective scattering capability of the contrast agent microbubbles. The increase in scattering capability results because the ultrasound scattering is a function of the contrast agent microbubble size. The scattering increases when the contrast agent microbubbles are about equal and/or above the resonance size. Hence, an increase in the level of the second harmonic signal from the contrast agent microbubbles is achieved by the introduction of modification pulse 404 between the first imaging pulse 402 and second nonlinear imaging pulse 406. It should be noted that in various embodiments a plurality of modification pulses 404 may be transmitted between each of a plurality of imaging pulses 402 and 406.

Modification pulse 404 is configured to change any acoustic and/or physical properties of the microbubbles and is not limited to the changes described above. For example, the modification pulse 404 may be configured to change one or more surface properties of the microbubbles. However, again, modification pulse 404 does not change, for example, the acoustic properties of the tissue in a human body. In the complete process of transmitting imaging and modification pulses and receiving echo signals, two images are generated by the two echo signals in response to the two nonlinear imaging pulses. One of the differences in these two obtained images is in the area where contrast agent microbubbles are present.

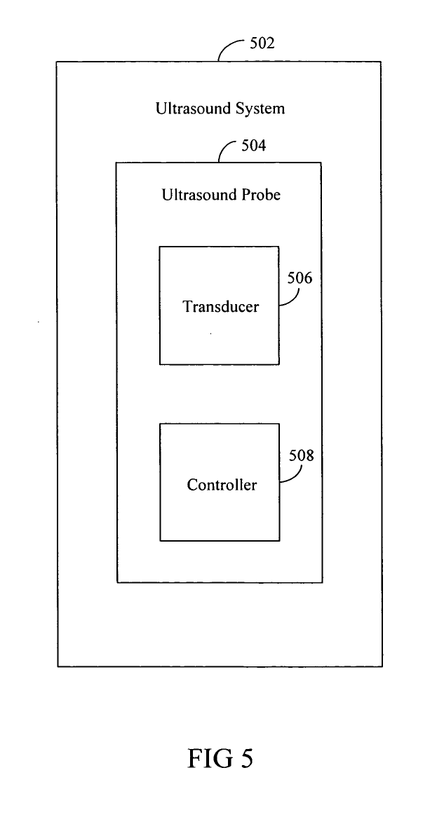

FIG. 5 is a block diagram of an exemplary embodiment of an ultrasound system according to various embodiments to the present invention for transmitting modification pulses as described herein. Ultrasound imaging system 502 is used for generating images from the received echo signals. Ultrasound imaging system 502 includes an ultrasound probe 504, which has a transducer 506, and a controller 508. Transducer 506 is utilized by ultrasound probe 504 for transmitting pulses to an object (e.g., human body or a part thereof). Controller 508 is configured to transmit at least one modification pulse, for example, between successive pulses in the nonlinear imaging pulse sequence as described herein. In another embodiment of the present invention, controller 508 will be located and operated from outside ultrasound imaging system 502. In another embodiment, controller 508 is located and operated from a remote location outside ultrasound imaging system 502.

Various embodiments of the present invention may be used to generate ultrasound images of the body parts using a modification pulse enhanced technique that allows for enhancement of the pulse-echo ultrasound energy returned by targeted tissue or blood flow containing microbubbles of the ultrasound contrast agents. Backscattering of signals from the contrast agent microbubbles is also improved with minimal disturbance to the tissue, thereby providing improved contrast. Further, the imaging techniques described herein enhance the sensitivity of contrast imaging. An increase in the contrast-to-tissue ratio is provided. Thus, various embodiments of the present invention use the difference in the intensities of harmonics generated and reflected by tissue and microbubbles in response to a sequence of nonlinear imaging pulses to improve imaging. It is noted that the imaging and modification pulse sequences may be transmitted along the same vector or different vectors.

The various embodiments of systems as described herein and any of their components, may be embodied in the form of a computer system. Typical examples of a computer system include a general-purpose computer, a programmed microprocessor, a micro-controller, a peripheral integrated circuit element, and other devices or arrangements of devices that are capable of implementing the process described herein.

The computer system may include, for example, a computer, an input device, a display unit and a communication interface, for example, for communicating with the Internet. The computer includes a microprocessor, with the microprocessor connected to a communication bus. The computer also includes a memory. The memory may include Random Access Memory (RAM) and Read Only Memory (ROM). The computer system further includes one or more storage devices. The storage devices may be a hard disk drive or a removable storage drive, such as, for example, a floppy disk drive, optical disk drive, and the like. The storage devices also may be other similar means for loading computer programs or other instructions into the computer system.

The computer system executes a set of instructions that are stored in one or more storage elements in order to process input data. The storage elements also may hold data or other information as desired or needed. The storage element may be in the form of an information source or a physical memory element present in the processing machine.

The set of instructions may include various commands that instruct the processing machine to perform specific tasks such as the processes or the various embodiments described herein. The set of instructions may be in the form of a software program. The software may be in various forms such as, for example, system software or application software. Further, the software may be in the form of a collection of separate programs, a program module within a larger program or a portion of a program module. The software also may include modular programming in the form of object-oriented programming. The processing of input data by the processing machine may be in response to user commands, or in response to results of previous processing or in response to a request made by another processing machine.

While the invention has been described in terms of various specific embodiments, those skilled in the art will recognize that the invention can be practiced with modification within the spirit and scope of the claims.

1. A method for controlling an ultrasound system, said method comprising:
transmitting a nonlinear contrast imaging pulse sequence; and
transmitting at least one modification pulse between successive pulses in the nonlinear contrast imaging pulse sequence, the modification pulse configured to change a contrast agent within an object being imaged by an ultrasound system.

2. A method in accordance with claim 1 wherein the contrast agent comprises at least one contrast microbubble.

3. A method in accordance with claim 1 wherein the contrast agent comprises at least one contrast microbubble and the modification pulse is configured to change a scattering cross-section of the at least one contrast microbubble.

4. A method in accordance with claim 1 further comprising receiving echo signals in response to the successive pulses, the received echo signals providing information for imaging using the ultrasound system.

5. A method in accordance with claim 1 further comprising receiving echo signals in response to the successive pulses and the modification pulse, the received echo signals providing information for imaging using the ultrasound system.

6. A method in accordance with claim 1 wherein the transmitting comprises transmitting a plurality of modification pulses between successive pluralities of pulses in the imaging pulse sequence.

7. A method in accordance with claim 1 wherein the object is a human body and the modification pulse is configured to change the acoustic property of the contrast agent and not the acoustic property of tissue within the human body.

8. A method in accordance with claim 1 wherein the imaging pulse sequence comprises a pulse-inversion imaging pulse sequence.

9. A method in accordance with claim 1 wherein the imaging pulse sequence comprises a plurality of pulses and inverse pulses.

10. A method in accordance with claim 1 wherein the modification pulse is configured to increase a level of a second harmonic signal from the contrast agent.

11. A method in accordance with claim 1 wherein the modification pulse is configured to increase a level of a nonlinear fundamental or first harmonic signal from the contrast agent.

12. A method in accordance with claim 1 wherein the contrast agent comprises at least one contrast microbubble and the modification pulse is configured to modify the at least one microbubble to a resonant dimension.

13. A method in accordance with claim 1 wherein the contrast agent comprises at least one contrast microbubble and the modification pulse is configured to modify a resonance frequency of the at least one contrast microbubble to substantially an imaging frequency of the ultrasound system.

14. A method in accordance with claim 13 wherein the modification comprises a shift in the resonance frequency of the contrast microbubble.

15. A method in accordance with claim 1 wherein a pulse in the nonlinear contrast imaging pulse sequence transmitted before the modification pulse is different than a pulse in the nonlinear contrast imaging pulse sequence transmitted after the modification pulse.

16. A method in accordance with claim 15 wherein the pulse transmitted before the modification pulse and the pulse transmitted after the modification pulse are inverted.

17. A method in accordance with claim 1 further comprising using for imaging information received in response to the modification pulse.

18. A method in accordance with claim 1 wherein the contrast agent comprises at least one contrast microbubble and the modification pulse is configured to change a scatterability of the at least one contrast microbubble.

19. A method in accordance with claim 1 wherein the contrast agent comprises at least one contrast microbubble and the modification pulse is configured to change at least one of (i) a surface property and (ii) a size of the at least one contrast microbubble.

20. A method in accordance with claim 1 further comprising comparing echo information received in response to the successive pulses to generate an image using the ultrasound system.

21. A method in accordance with claim 1 further comprising comparing echo information received in response to the successive pulses and modification pulse to generate an image using the ultrasound system.

22. A method in accordance with claim 1 wherein the modification pulse is configured to change a physical property of the contrast agent.

23. A method in accordance with claim 1 wherein the modification pulse is configured to change an acoustic property of the contrast agent.

24. A method for contrast imaging with an ultrasound system, said method comprising:
transmitting at least one modification pulse between successive pulses in an imaging pulse sequence, the modification pulse configured to change a contrast agent within an object being imaged by an ultrasound system;
receiving image data in response to transmission of the imaging pulse sequence; and
using the received image data to generate an image with the ultrasound system.

25. A method in accordance with claim 24 further comprising receiving image data in response to transmission of the at least one modification pulse and using the received image data to generate an image with the ultrasound system.

26. A method in accordance with claim 24 wherein the transmitting comprises transmitting a plurality of modification pulses between successive pluralities of pulses in the nonlinear contrast imaging pulse sequence.

27. A method in accordance with claim 24 wherein the contrast agent comprises at least one microbubble.

28. A method in accordance with claim 24 wherein the nonlinear contrast imaging pulse sequence comprises a pulse-inversion imaging pulse sequence.

29. An ultrasound system comprising:
an ultrasound probe having at least one transducer for transmitting pulses to an object; and
a controller configured to transmit at least one modification pulse between successive pulses in a nonlinear contrast imaging pulse sequence, the modification pulse changing a contrast agent within an object being imaged by the ultrasound probe.
